(19) United States
(12) Patent Application Publication
(10) Pub. No.: US 2025/0186800 A1
(43) Pub. Date: Jun. 12, 2025

(54) LIGHT-FIBER CATHETER WITH CALIBRATED OUTPUT AND METHODS

(71) Applicant: ALUCENT BIOMEDICAL INC.

(72) Inventors: James D. ISAACSON, Salt Lake City, UT (US); RB Eugene HAYES, Salt Lake City, UT (US); Dylan James BLAIR, Salt Lake City, UT (US)

(21) Appl. No.: 18/532,414

(22) Filed: Dec. 7, 2023

(51) Int. Cl. A61N 5/06; A61L 2/08; A61N 5/067

(52) U.S. Cl. CPC A61N 5/062; A61L 2/087; A61N 5/0601; A61N 5/0624; A61L 2202/24; A61N 2005/0602; A61N 2005/063; A61N 5/067

(57) ABSTRACT

Systems, methods, and devices are disclosed for a catheter device. The catheter device may include a catheter body having a proximal and a distal end. The catheter body may have a light fiber for activating a natural vascular scaffolding (NVS) agent. The one or more light fibers may extend distally from the proximal end to an emission zone. The catheter may include a first section of the light fiber comprised of a first material extending a first length. The catheter may include a second section of the light fiber comprised of a second material extending a second length, wherein the second material is different than the first material. The catheter may include an emission zone disposed proximal to the distal end.

### TECHNICAL FIELD

The present disclosure relates to devices, systems, and methods for varying light outputs for Natural Vascular Scaffolding (NVS).

### SUMMARY

Disclosed embodiments include systems, methods, and apparatus for a catheter device. Some disclosed embodiments involve a catheter device having a catheter body. In some embodiments, the catheter body may have a proximal and a distal end. In some embodiments, the catheter body may have a light fiber for activating a natural vascular scaffolding (NVS) agent, the one or more light fibers extending distally from the proximal end to an emission zone. Some disclosed embodiments involve a first section of the light fiber having a first material extending a first length. A catheter device may include a second section of the light fiber having a second material extending a second length, where the second material may be different than the first material. In some embodiments, the emission zone may be disposed proximal to the distal end.

In some embodiments, the first material may include silica. In some embodiments, the second material may include Polymethyl Methacrylate (PMMA). In some embodiments, the emission zone may be disposed within the second section. In some embodiments, the device may be configured for electron beam sterilization. In some embodiments, a change of the first length or the second length modulates a light output. In some embodiments, the emission zone may be configured so that, in use, light output activates the natural vascular scaffolding agent. In some embodiments, the first section and the second section abut each other. Some disclosed embodiments include a third section extending a third length, where the third section may include a third material, where the third material may be different than the second material. Some disclosed embodiments include a light source for the light output, wherein the light source may be coupled to the proximal end.

Disclosed embodiments include a method for configuring a light output of a light fiber device. Some disclosed embodiments involve determining for a light fiber for activating a natural vascular scaffolding (NVS) agent, a first length of a first section comprised of a first material and a second length of a second section comprised of a second material. Some disclosed embodiments involve assembling a catheter device including a body having: a proximal and a distal end and the light fiber, the light fiber extending distally from the proximal end to an emission zone. In some disclosed embodiments, the second material may be different than the first material. In some disclosed embodiments, the emission zone may be disposed proximal to the distal end. Some disclosed embodiments involve sterilizing the catheter device with electron beam sterilization, wherein the determined first length or the determined second length varies a light output of the light fiber.

In some embodiments, the first material includes a material not affected by electron beam sterilization. In some embodiments, the first material includes silica. Some disclosed embodiments involve increasing the first length, wherein increasing the first length reduces a loss of light output after electron beam sterilization. In some embodiments, the second material comprises Polymethyl Methacrylate (PMMA). Some disclosed embodiments involve increasing the second length, wherein increasing the second length reduces the light output. In some disclosed embodiments, reducing the light output modulates the activation of the natural vascular scaffolding. In some embodiments, the emission zone may be disposed within the second section. In some embodiments, the first section and the second section abut each other. Some disclosed embodiments involve a third section extending a third length, wherein the third section may be comprised of a third material, wherein the third material may be different than the second material.

Some disclosed embodiments include a catheter device including a catheter body having a light fiber for activating a natural vascular scaffolding (NVS) agent by a light output, the one or more light fibers extending distally from a proximal end to an emission zone. Some disclosed embodiments include a first section of the light fiber having a first diameter; and a second section of the light fiber having a second diameter. In some disclosed embodiments, the second diameter is different than the first diameter. In some disclosed embodiments, a difference between the first diameter and the second diameter may be predetermined to modulate a predicted loss of the light output.

In some disclosed embodiments, the first diameter is larger than the second diameter. In some embodiments, an increase in the difference between the first diameter and the second diameter increases the loss of the light output. In some embodiments, the first section may be disposed proximal to a light source coupled to the proximal end. In some embodiments, the second section may be disposed proximal to a distal end. In some embodiments, the first section may be comprised of a first material and the second section may be comprised of a second material. In some embodiments, the first material may be different than the second material.

In some embodiments, the first material comprises silica. In some embodiments, the second material comprises Polymethyl Methacrylate (PMMA). In some embodiments, the first section and the second section abut each other. In some embodiments, the loss of the light output occurs at a junction between the first section and the second section.

Some disclosed embodiments include a method for configuring a light output of a light fiber device. Some disclosed embodiments involve determining, for a light fiber for activating a natural vascular scaffolding (NVS) agent by a light output, a first diameter of a first section of the light fiber and a second diameter of a second section of the light fiber, wherein the second diameter may be different than the first diameter; Some disclosed embodiments include assembling a catheter device comprising: a body having a proximal end; and the light fiber extending distally from a proximal end to an emission zone. In some embodiments, the determined first diameter and the determined second diameter modulate a predicted loss of the light output.

In some embodiments, the first diameter may be greater than the second diameter. Some disclosed embodiments involve increasing the loss of the light output by increasing the difference between the first diameter and the second diameter increases the loss of the light output. In some embodiments, the first section may be disposed proximal to a light source coupled to the proximal end. In some embodiments, the second section may be disposed proximal to a distal end. In some embodiments, the first section may be comprised of a first material and the second section may be comprised of a second material. In some embodiments, the first material may be different than the second material. In some embodiments, the first material comprises silica. In some embodiments, the second material comprises Polymethyl Methacrylate (PMMA). In some embodiments, the first section and the second section abut each other. In some embodiments, the loss of the light output occurs at a junction between the first section and the second section.

Some disclosed embodiments include a catheter device comprising a catheter body having a light fiber for activating a natural vascular scaffolding (NVS) agent by a light output, the light fiber extending distally from a proximal end to an emission zone. In some embodiments, the light fiber may be comprised of a material configured for electron beam sterilization. In some embodiments, a diameter of the light fiber may be predetermined to modulate a predicted reduction in transmission of the light output by the electron beam sterilization.

In some embodiments, an increase in the diameter of the light fiber increases the reduction in transmission of the light output by the electron beam sterilization. In some embodiments, a decrease in the diameter of the light fiber reduces the reduction in transmission of the light output by the electron beam sterilization. In some embodiments, the material comprises Polymethyl Methacrylate (PMMA). Some disclosed embodiments involve a light source coupled to the proximal end. In some embodiments, the emission zone is configured so that, in use, the light output activates the natural vascular scaffolding agent. In some embodiments, the reduction in transmission of the light output increases after a time period.

Some disclosed embodiments include a method for configuring a light output of a light fiber device. Some disclosed embodiments involve determining a diameter of a light fiber for activating a natural vascular scaffolding (NVS) agent by a light output. Some disclosed embodiments involve assembling a catheter device comprising a body having: a proximal end; and the light fiber, the light fiber extending distally from a proximal end to an emission zone, wherein the light fiber is comprised of a material configured for electron beam sterilization. Some disclosed embodiments involve applying the electron beam sterilization to the light fiber, wherein the determined diameter of the light fiber modulates a predicted reduction in transmission of the light output by the electron beam sterilization.

In some embodiments, increasing the diameter of the light fiber increases the reduction in transmission of the light output by the electron beam sterilization. In some embodiments, decreasing the diameter of the light fiber reduces the reduction in transmission of the light output by the electron beam sterilization. In some embodiments, the material comprises Polymethyl Methacrylate (PMMA). Some disclosed embodiments involve a light source coupled to the proximal end. In some embodiments, the emission zone is configured so that, in use, the light output activates the natural vascular scaffolding agent. In some embodiments, the reduction in transmission of the light output increases after a time period.

### BACKGROUND

Traditional or conventional systems for treating narrowed blood vessels may involve the use of physical stents, such as metal or mesh stents. However, such angioplasty methods may not be effective in preventing restenosis in some applications. Moreover, treatment of arteriovenous fistula, including enhancing the maturation of surgically-placed arteriovenous fistula (AVF), may require improvements over traditional or conventional systems. For example, an AVF may be surgically created by connecting a vein to an artery to enable vascular access for hemodialysis (e.g., for renal conditions). In some cases, AVF maturation failure can be caused by luminal stenosis due to excessive neointimal hyperplasia and/or impaired outward remodeling. AVF maturation can be unsuccessful not only if the vessel wall does not thicken, but also if vessel wall thickening is accompanied by a decrease in lumen size. If maturation fails, the surgical procedure must be repeated. As such, it is desirable to promote maturation and simultaneously promote the success of the AVF. It will be recognized that there is a need for improvements for venous treatments and procedures, including strengthening venous walls and maximizing the open lumen area.

Natural Vascular Scaffolding (NVS), involves using a photoactivatable drug and a wavelength of light to cross link extracellular matrix proteins, presenting a potent alternative to traditional venous procedures, including traditional stenting. NVS can also present enhancements to the promotion of AVF maturation. Natural Vascular Scaffolding may involve catheters including light fibers and balloons for activating the therapy agents for NVS. Since NVS therapy can utilize different amounts of light for activating various NVS agents, there is a need for varying the amount of light delivered to the NVS agent, including the amount of light transmitted from a light source to the NVS agent through a light fiber. Adjusting the output of light or the transmission of light in light fibers may involve varying the power source, such as adjusting the power output of a source. However, such methods of adjustment may be prone to user error, such as selecting a wrong device type on a laser light source, and such methods may also involve expensive capital equipment costs and increased item costs.

Moreover, the use of catheters may present complications if such catheters are sterilized, as various components may be affected differently by the sterilization process. Separate sterilization processes for a catheter and any drug used for the treatment, as well as sterilization of a light fiber, may be time consuming and expensive. Further, an integrated catheter device (e.g., a device including a catheter and a light fiber assembled before use) may be preferable for a clinician. For example, a surgeon may prefer a device that integrates a light fiber and a balloon catheter before sterilization of the device and/or before clinical use in comparison to a device that has a separate catheter and light fiber, which may not be ideal. Thus, there is a need for an integrated catheter device for improvements to venous treatments and procedures.

### BRIEF DESCRIPTION OF DRAWINGS

FIG. 1 is an illustration of a catheter device, consistent with embodiments of the present disclosure.

FIG. 2 is an illustration of a light fiber, consistent with embodiments of the present disclosure.

FIG. 3 is an illustration of a light fiber, consistent with embodiments of the present disclosure.

FIG. 4 is an illustration of a light fiber, consistent with embodiments of the present disclosure.

FIG. 5 is an illustration of a light fiber, consistent with embodiments of the present disclosure.

FIG. 6 is an illustration of an exemplary flow diagram of a method for configuring a light output, consistent with embodiments of the present disclosure.

FIG. 7 is an illustration of a light fiber, consistent with embodiments of the present disclosure.

FIG. 8 is an illustration of a light fiber, consistent with embodiments of the present disclosure.

FIG. 9 is an illustration of a light fiber, consistent with embodiments of the present disclosure.

FIG. 10 is an illustration of an exemplary flow diagram of a method for configuring a light output, consistent with embodiments of the present disclosure.

FIGS. 11A-11B are graphs of exemplary light attenuation in a light fiber, consistent with embodiment of the present disclosure.

FIGS. 12A-12B illustrate graphs of the effect of core diameter, consistent with embodiments of the present disclosure.

FIG. 13 illustrates an exemplary flow diagram of a method for configuring a light output, consistent with embodiments of the present disclosure.

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that some embodiments may be utilized and that changes may be made without departing from the scope of the disclosed embodiments. Thus, the materials, methods, and examples are illustrative only and are not intended to be necessarily limiting.

Natural Vascular Scaffolding (NVS) therapy may refer to treatment of a substrate using at least one active agent according to the present disclosure. NVS may involve broadening certain vessels or openings in the body which may have been narrowed due to disease or other conditions. As a non-limiting example, NVS may have applications in peripheral artery disease, vascular injury, vascular dissection, coronary artery disease, fistula maturation, fistula repair, venous disease, carotid and renal artery interventions, and prevention of abdominal aortic aneurysm progression. NVS may interlink collagen and elastin by covalently linking these proteins via photoactivation. Catheters can be employed to deliver NVS compounds to a treatment site or treatment location within the body. These catheters can also include a light fiber to deliver light to the treatment site for photoactivation of the NVS agent.

As described herein, it may be desired to adjust, vary, or modulate the output of light at an emission zone for procedures involving catheters and light fibers, such as NVS therapies. It may be desired to vary the light output to provide appropriate output for different clinical applications, such as different balloon diameters for treatments of conditions affecting blood vessels. The light may be transmitted through the light fiber from a light source to an emission zone, which may be where the NVS therapy occurs (e.g., a balloon coated with photoactivatable small molecules) In some examples, certain devices may need lower light outputs, as in the example of balloons with smaller diameters. It will be recognized that it may be expensive or error-prone to vary light outputs at the light source. For example, changing the power output of the light source may involve increased costs. It would also be more efficient to sterilize the catheter and light fiber together after they have been joined. Thus, there exists a need for a catheter device including an integrated light fiber to address conditions affecting blood vessels. As such, disclosed embodiments may involve catheters enabling the variation of light output of light fibers, including for applications of Natural Vascular Scaffolding (NVS). Disclosed embodiments involve modifying the light fiber of a catheter itself to affect the transmission of light in the fiber, thereby affecting the light output. The disclosed embodiments may involve intentionally inducing inefficiencies in the light fiber in order to deliver a desired output to the vessel without adjusting the laser power at the source. Thereby, different light fibers may be tuned to provide different light outputs suitable for different clinical applications without modification of the laser source.

Light fibers as described herein may include light in any wavelength which can enable activation of a NVS agent, such as 450 nanometer wavelength light as a non-limiting example. Other wavelengths are possible, including both visible and invisible light, according to the NVS agent formulation. Light sources may include source of light, including lasers. The methods disclosed herein include illuminating the at least one active agent with visible light. For example, the light may activate the NVS agent in a diffusion zone including a balloon.

Some disclosed embodiments may also involve sterilization of the catheter, including electron beam sterilization. For example, sterilization may be used for pre-packaged devices, such as catheters where sterile packaging is opened before clinical use. Electron beam (e-beam) sterilization may use an electron beam to ionize the surface of a material to sterilize it, including for medical purposes such as elimination of bacteria or contaminants. Sterilization may affect the properties of light fibers, and thereby affect the transmission of the light in the light fibers. As such, in some examples, sterilization may be a consideration for varying the output of light.

Disclosed embodiments may include a catheter device. Catheters may refer to flexible tubes inserted through openings in the body (e.g., lumens) including blood vessels, such as veins or arteries. Catheters may extend from a point of insertion outside the body, such as the groin or neck, to a target delivery location, such as a blood vessel. Catheters may assist in the treatment of conditions affecting blood vessels, including for treating narrowed blood vessels (e.g., opening or broadening narrowed blood vessels, such as by stents or other methods described herein). In some embodiments, a catheter may have a proximal end and a distal end. A proximal end may refer to a portion or side of a catheter close to a point of insertion. For example, the proximal end may refer to the end of a catheter device which may be close to a light source. A distal end may refer to a portion or side opposite the point of insertion, such as the tip of a catheter which may be opposite the proximal end. In some examples, the distal end may include materials which may be delivered to a target location, such as a balloon (e.g., an angioplasty balloon) and treatment sources (e.g., drugs, small-molecule compounds).

Some disclosed embodiments involve a light fiber. A light fiber may refer to any light-conducting fiber device for transmitting signals, such as transmitting light across a distance. Light fibers may transmit electromagnetic radiation, including various frequencies of light (e.g., including but not limited to infrared, visible, and ultraviolet light). For example, light fibers may transmit light from a proximal end to a distal end. Light fibers may enable signals to travel with minimal losses or attenuation of the signal. Light fibers may also refer to optical fibers or fiber-optic cables. Light fibers may be comprised of various materials and coated or cladded with various materials which may influence the transmission of the light. In some embodiments, light fibers may be comprised of materials such as glass and/or plastics. In some embodiments, the catheter device includes one or more light fibers. For example, a single catheter may contain multiple light fibers for transmission of a single signal or multiple signals.

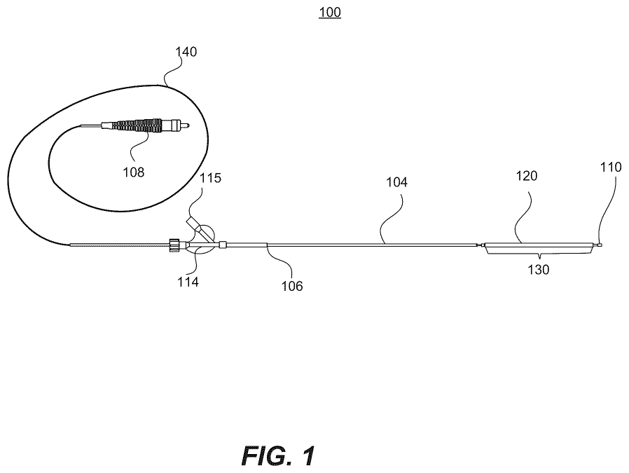

FIG. 1 illustrates a catheter device 100 in accordance with embodiments of the present disclosure. The catheter device 100 may include a catheter body having a light fiber 140. The catheter device 100 may be configured for longitudinal movement and positioning within a vessel (e.g., blood vessel) of a subject. The catheter device 100 may include a proximal end connector 114 positioned at the proximal end of the catheter device 100, and the catheter shaft 104 may extend in a distal direction therefrom. The catheter shaft 104 incorporation with the light fiber 140 may be a plurality of lumens that are accessible via a plurality of ports the proximal end connector 114. The plurality of ports 115 may be configured to engage with external sources desirable to communicate with the plurality of lumens. The ports may engage with external sources via a variety of connection mechanisms, including, but not limited to, syringes, over-molding, quick disconnect connectors, latched connections, barbed connections, keyed connections, threaded connections, or any other suitable mechanism for connecting one of the plurality of ports to an external source. Non-limiting examples of external sources may include inflation sources (e.g. saline solutions), gaseous sources, treatment sources (e.g. medication, drugs, or any desirable treatment agents discussed further below), light sources, among others. In some embodiments, catheter device 100 can be used with a guide wire (not shown), via guide wire lumen (not shown), to assist in guiding the catheter shaft 104 to the target area of the vessel.

In some embodiments, catheter device 100 may include a light fiber 140 incorporated into a catheter, forming an integrated catheter and light fiber device. A catheter body may have one or more optically sufficient performing light fibers 140. The light fibers may form a light path of the catheter shaft body. As such, catheter device 100 may involve an integrated device which can utilize common and/or shared sterilization techniques suitable for the light fiber, catheter, and/or any drugs used for treatment (e.g., NVS agents).

The catheter device 100 may include a distal balloon 120 positioned over a distal segment 130 of the catheter shaft 104 proximal to the distal tip 110. The distal balloon 120 may take any shape suitable for supporting a wall of a blood vessel of the subject when the non-compliant or semi-compliant balloon is inflated. For example, the distal balloon 120 may expand into a cylindrical shape surrounding the distal segment 130 of the catheter shaft 104. In some embodiments, the distal segment 130 may include an emission zone. For example, catheter device 100 may include one more emission zones, including an emission zone disposed within a region of catheter device 100 covered by the distal balloon 120.

In some embodiments, catheter device 100 may be coupled to a light source. For example, catheter device 100 may include a connector (e.g., adapter) for a light source, such as connector 108. Connector 108 may be coupled to a proximal end of catheter device 100 (such as a proximal end of light fiber 140) and may couple light fiber 140 to any light source, including an external light source (e.g., a laser source). In some examples, connector 108 may be a removably coupled connector, such as a coaxial radio frequency connector (e.g., a SubMiniature version A connector).

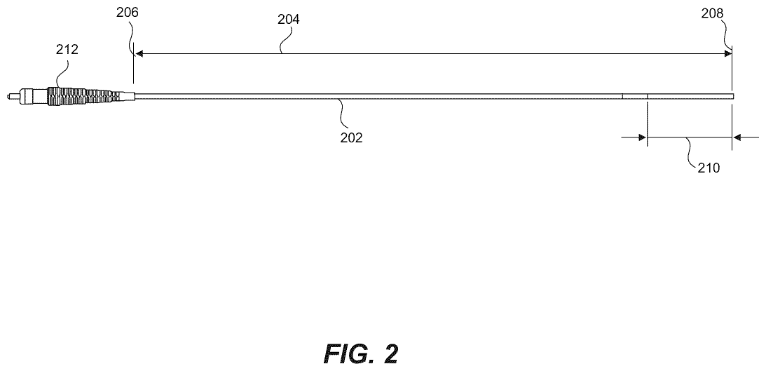

FIG. 2 illustrates a light fiber, consistent with embodiments of the present disclosure. Light fiber 200 may include a fiber optic cable 202 (e.g., a wire) extending distally from a proximal end 206 to an emission zone 210. Extending distally may involve extending from a proximal side toward a distal side, such as having a length which travels from a proximal end 206 towards a distal end 208. Light fiber 200 may transmit signals including light originating from an external source (not illustrated in FIG. 2) connected or coupled to light fiber 200 by a 212. Fiber optic cable 202 may transmit, propagate, or otherwise send light along the length 204 of the light fiber. For example, light fiber 200 may enable light to travel from light source connector 212 to emission zone 210. The connector 212 may be coupled to a proximal end 206, such that light may be transmitted from a light source attached to the connector 212 to the fiber optic cable 202. In some embodiments, emission zone 210 may be disposed proximal to distal end 208, such that the emission zone may be delivered to the target tissue or blood vessel location. Emission zone 210 may be an area, region, or component of light fiber 200 for emitting or releasing signals transmitted in light fiber 200. For example, emission zone 210 may be a region of light fiber 200 which includes a balloon and/or one or more drugs. A balloon may be disposed within emission zone 210 such that the balloon surrounds the fiber optic cable 202. Emission zone 210 may also include a light diffuser or any method of spreading the light. In some examples, an angioplasty balloon in the emission zone 210 may be coated with drugs such as a Natural Vascular Scaffolding (NVS) agent. In some examples, light fiber 200 may include external sources, such as inflation sources (e.g. saline solutions), gaseous sources, treatment sources (e.g. medication, drugs, or any desirable treatment agents), and light sources, as non-limiting examples of sources which may be coupled or attached to the catheter. In some embodiments, light fiber 200 may include a guide wire or cable.

It will be recognized that light fiber 200 may be comprised of one or more materials. In some embodiments, fiber optic cable 202 may be comprised of any material suitable for transmission of signals including light. For example, light fiber 200 may include one or more materials such as glass, silica (e.g., silicon dioxide), quartz, polymers (e.g., nylon), PEBAX, HDPE, LDPE, polyethylene, polyurethane, or a composite material of plastic with a reinforcement (e.g., metal wires, metal coil, plastic fibers). It will also be recognized that various factors, including the material composition of light fiber 200, may affect the transmission of the light in the light fiber, and thereby affect the output of light in the emission zone 210. For example, various factors may affect the transmission of light in light fiber 200 through absorption, scattering, reflection, and/or diffraction, thereby contributing to attenuation of the light (e.g., transmission loss). Additional factors including optical power and dispersion may also affect attenuation. As such, it will be appreciated that by utilizing different materials in light fiber 200, the attenuation of the light, and thereby the amount of light output available at the emission zone 210, may be modulated to deliver the appropriate amount of light to a therapeutic target area.

In some examples, as described herein, light fibers may experience electron beam sterilization, which can affect various materials and may induce material changes (e.g., physical, mechanical, chemical) in the materials. A light fiber configured for electron beam sterilization may refer to a light fiber including material(s) which may have such material changes during or after the application of electron beam sterilization. For example, light fiber 200 may include Polymethyl Methacrylate (PMMA), and the fiber optic cable 202 may be made of the PMMA extending a length 204. Materials such as PMMA may enable outward diffusing zones for drugs (e.g., photoactivatable agents). It will be recognized that the electron beam sterilization may affect the PMMA and cause material changes in the PMMA which can reduce the transmission of the light (e.g., causing light outputting and/or performance losses in the PMMA). For example, for a light fiber having a length 204 of PMMA, there may be an expected loss of up to 95% of the light output after electron beam sterilization, depending on the core diameter of the fiber (e.g., 95% of the transmitted light or the amount of light originating from the light source may not be transmitted to the emission zone 210). Thus, in some disclosed embodiments, it will be appreciated that by modulating the amount of materials affected by electron beam sterilization, the expected loss of the output of the light may be modulated. For example, by increasing or decreasing the amount of PMMA in the light fiber, as well as increasing or decreasing the amount of other materials which the light fiber can be made of, the expected loss of the output of the light may be predetermined and increased or decreased.

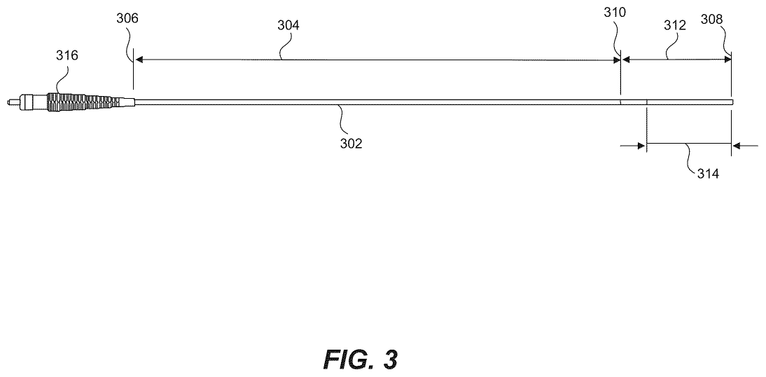

FIG. 3 illustrates a light fiber, consistent with embodiments of the present disclosure. Light fiber 300 may include one or more fiber optic cables 302, which may be made of one or more materials spanning one or more sections (e.g., segments). For example, light fiber 300 may include a first section extending a first length 304, and a second section extending a length 312. The first section may be comprised of a first material, and the second section may be comprised of a second material. In some embodiments, the second material (e.g., the material in the second section) may be different than the first material (e.g., the material in the first section). For example, the first material may be a material not affected (e.g., does not experience substantial material changes) after electron beam sterilization, and the second material may be a material which can be affected by electron beam sterilization, or vice-versa. In some embodiments, the first material in the first section includes silica, and the second material in the second section includes PMMA. For example, the PMMA in light fiber 300 may extend a length 312, and the silica may extend a length 304. In some embodiments, the first section may be located adjacent to the proximal end 306, and the second section may be located adjacent to the distal end 308. In some embodiments, the first section and the second section may abut each other. For example, the silica in second section may contact the PMMA in the second section at a contact point 310. In some embodiments, emission zone 314 may be disposed within the second section, and light fiber 300 may transmit the light from a light source through the connector 316 to the emission zone 314.

It will be appreciated that changing the first length 304 and/or the second length 312 may involve increasing or decreasing the amount of the first material in the first section as well as increasing or decreasing the amount of the second material in the second section, thereby modulating the amount of light output, as described herein. The length 304 of the first section can also be changed while the length 312 of the second section may be fixed, and vice-versa, such that the length 304 of the first section can be fixed while the length 312 of the second section changes. It will be appreciated that changing the length different materials span along the length of the fiber optic cable 302 (e.g., for the same core diameter) may enable a predetermined expected loss post electron beam sterilization. In an example in which silica is the first material, since the silica is not affected by electron beam sterilization, maximizing or increasing the amount of silica can result in a lower loss of light output post electron beam sterilization (e.g., more light is transmitted to the emission zone 314), thereby accounting for losses in the PMMA. For example, silica spanning a first length f304 and PMMA spanning a second length 312 may result in a 15% expected loss of light output post electron beam sterilization.

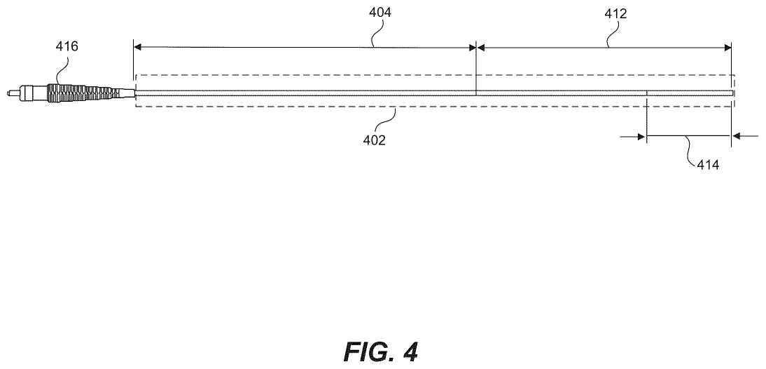

FIG. 4 illustrates an example of a light fiber, consistent with embodiments of the present disclosure. Light fiber 400 may include fiber optic cables 402, which may represent fiber optic cables 202 or fiber optic cables 302 (as referenced in FIG. 2 and FIG. 3, respectively) with changes to the lengths of the first section and the second section to achieve a desired light output amount. For example, fiber optic cable 402 may have a first section spanning a first length 404 and a second section spanning a second length 412. The first section may be comprised of silica, and the second section may be comprised of PMMA, as described herein. In an example, silica extending a length 404 and PMMA extending a length 412 may achieve a desired light output amount in the case of a 45% expected loss of light post electron beam sterilization. In some embodiments, emission zone 414 may be disposed within the second section, and light fiber 400 may transmit the light from a light source through the connector 416 to the emission zone 414.

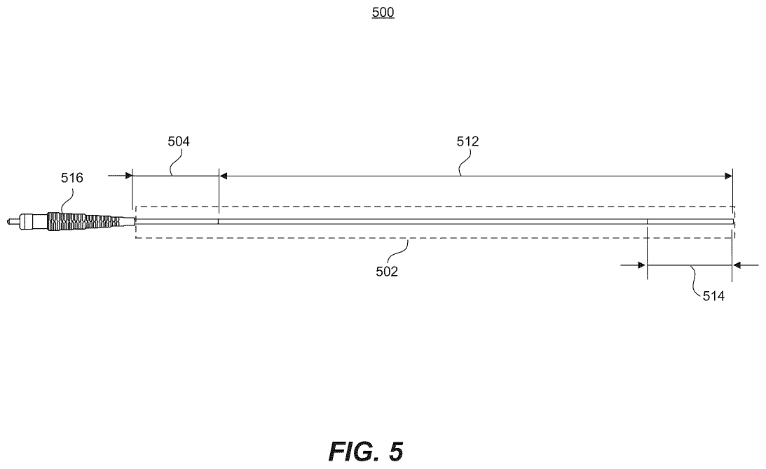

FIG. 5 illustrates an example of a light fiber, consistent with embodiments of the present disclosure. Light fiber 500 may include fiber optic cable 502, which may have a first section spanning a first length 504 and a second section spanning a second length 512. The first section may be comprised of silica, and the second section may be comprised of PMMA, as described herein. In an example, silica extending a length 504 and PMMA extending a length 512 (which may be a decrease in the length 404 of the silica and an increase in the length 412 of the PMMA as referenced in FIG. 4) may achieve a desired light output amount in the case of an 85% expected loss of light post electron beam sterilization (e.g., a more reduced light output than the configuration of FIG. 4). In some embodiments, emission zone 514 may be disposed within the second section, and light fiber 500 may transmit the light from a light source through the connector 516 to the emission zone 514.

In some embodiments, light fibers as discussed herein may be comprised of multiple sections or segments. In some embodiments, a light fiber may include a third section extending a third length, and the third section may be disposed next to the second section. The third material may be different than the second material, such as in an example where the second material may be PMMA and the third material may be silica. The third material may also be the same as the first material, such as in an example where the first section may be made of silica, the second section may be made of PMMA, and the third section made of silica. In another example, the third section may be made of a material different than both the materials of the first and the second sections. It will be appreciated that disclosed embodiments may involve other arrangements of additional sections of materials, including the light fiber having any number of sections with any combination of materials to achieve a desired light output. For example, four, five, or more sections may be included in a catheter device, and each section may be made of a material affected (or alternatively, not affected) by electron beam sterilization, and each section may be made of a material already included in the light fiber (or alternatively, a new material), to achieve a desired modification of the light output.

In some embodiments, configuring a light output may involve determining an amount of light needed for use. For example, configuring a light output may include determining the amount of light (e.g., intensity and/or intensity over time) and/or the wavelength of the light for a clinical use. The amount of light may be based on the needs of a specific NVS drug or agent, as well as properties of the tissue in the delivery area, such as when the amount of light may be changed according to the thickness of the blood vessel (e.g., more light may be needed for transmitting light through a thicker artery in comparison to a thinner vein). For example, determining the amount of light may include factors such as a minimum or threshold amount of light needed to activate the NVS agent to undergo a reaction, as well as any distance the light may need to travel from the light fiber (e.g., a the amount of light needed for a 2 mm diameter vessel may be less than a 7 mm vessel, as the light may not need to travel as far). Configuring the light output may also involve determining a maximum amount of light, as too much light can cause damage to a blood vessel in some examples. Further, the size of the emission zone and/or the balloon can affect the amount of light needed (e.g., doubling the length of the emission zone may cut the dosage per area of drug in half, thereby affecting the light needed), as well as the light source spot size (e.g., beam diameter such as a laser diameter). Based on these exemplary, non-limiting factors, the amount of light can be determined for a given clinical use, and the catheter device can be configured to deliver the appropriate light output by configuring losses into the light fiber such that the light fiber transmits the appropriate light output (e.g., at an emission zone).

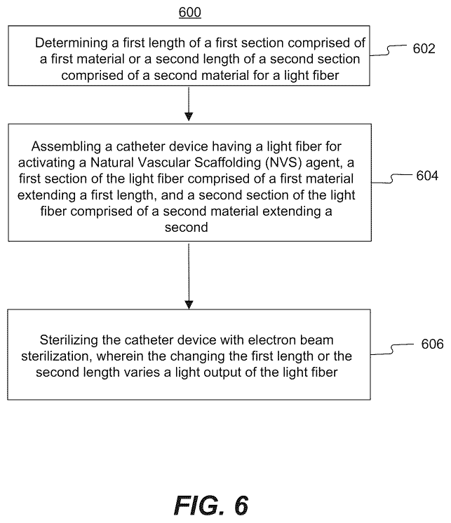

FIG. 6 illustrates an exemplary flow diagram of a method for configuring a light output, consistent with embodiments of the present disclosure. As described herein, a light fiber may include a first section comprised of a first material extending a first length and a second section comprised of a second material extending a second length. Method 600 may include a step 602 of determining a first length or the second length. The first length and/or the second length may be selected to achieve a desired light output. For example, the lengths may be determined experimentally (e.g., trial and error), as well as through various formulas and algorithms (e.g., methods which can account for transmission characterizations of the materials as a function of properties such as the length, core diameter, and sterilization dosage). Attenuation formulas, as described herein, can be utilized to approximate the attenuation based on the combination of materials and geometry of the materials (e.g., core diameter and length), and further experimentation could be used to refine any approximations. Step 602 may involve changing (e.g., increasing or decreasing) the first length while holding the second length constant, or changing the second length while holding the first length constant. In some examples, step 602 may involve changing both the first and the second length, or providing additional sections having different materials (e.g., providing a third section). Method 600 may include a step 604 of assembling or manufacturing a catheter device having a light fiber for activating a natural vascular scaffolding (NVS) agent, the light fiber extending distally from the proximal end to an emission zone. As described herein, assembling may refer to manufacturing, preparing, constructing, or the like. In some embodiments, the first section of the light fiber may include of a first material extending a first length, and a second section of the light fiber may include a second material extending a second length, and the second material may be different than the first material. In some examples, the emission zone may be disposed proximal to the distal end. In some embodiments, method 600 may include a step 606 of sterilizing the catheter device with electron beam sterilization. In some examples, changing the first length or the second length varies a light output of the light fiber. For example, after sterilization, the light output of the light fiber may be decreased, as described herein. In step 602, the first or second length may be changed to achieve an expected loss in the transmission of the light, and therefore a desired output of the light for a clinical application. In use, providing the catheter device may involve delivering the catheter device to the target location, such as a blood vessel or tissue. The NVS agent may be applied to the catheter device, including coating the catheter with the NVS agent (e.g., coating a balloon on the catheter with the drug). For example, in step 606, the emission zone may be configured so that, in use, the light output from the light fiber activates the natural vascular scaffolding agent. Step 606 may involve delivering the drug in the emission zone, which may involve inflating a balloon, such as expanding a balloon in a blood vessel (for example, expanding a narrowed blood vessel), enabling the NVS agent to diffuse into tissue, and transmitting light through the light fiber to the emission zone, thereby activating the NVS agent. Disclosed embodiments may involve extracellular matrix proteins crosslinking to promote physiological properties that hold the arterial lumen open or to promote venous remodeling, depending on the blood vessel of interest and/or the specified procedure. In an example, the light may activate the NVS agent to create fiber links in the tissue, such as links between collagen and elastin, thereby creating a natural scaffold which holds the lumen (opening) open. For example, a wavelength of light and intensity of light (e.g., a light dosage such as an intensity of light over a time period) In step 606, the balloon may be deflated, and the catheter may be removed.

It will be recognized that the size of light fibers, including the length of light fibers as well as the diameter of the light fiber, may affect the transmission of light in light fibers. For example, attenuation may be core-diameter dependent as well as material dependent. As such, it will be appreciated that by varying physical properties of the light fiber, including length and diameter, the transmission of the light may be modulated in order to achieve a desired expected light output.

In some embodiments, a light fiber may include different diameters of material. The diameter of a light fiber may refer to the diameter of the core of the light fiber. For example, one or more fibers (e.g., two fibers) may be joined together to form a light fiber, and the fibers may have different diameters. Two fibers can be joined together, and the fibers may be of different material or the same material. As an illustrative example, light fibers may have core diameter ranging from 250 micrometers to 750-1500 micrometers.

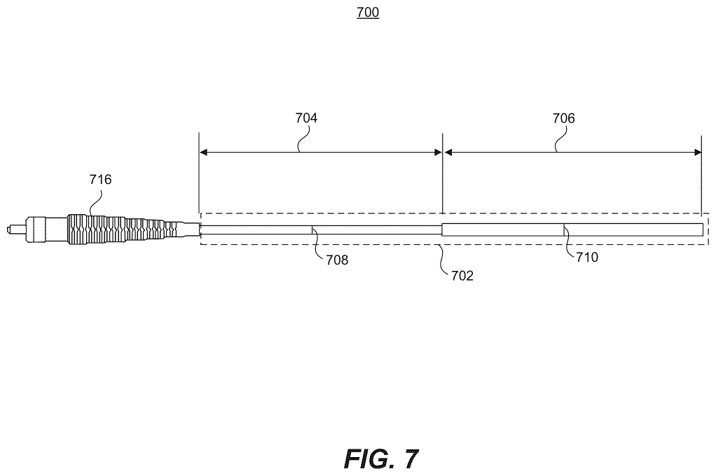

FIG. 7 illustrates an example of a light fiber, consistent with embodiments of the present disclosure. Light fiber 700 may include a fiber optic cable 702 having a first section 704, which may be disposed proximal to a connector 716 to a light source, and a second section 706, which may be disposed distal to the light source connector 712. In some examples, the first section 704 may abut the second section 706. The first section 704 may have a first diameter 708, and the second section 706 may have a second diameter 710. The first diameter 708 may be different than the second diameter 710, or the same as second diameter 710. In an example, the first diameter 708 may be smaller than the second diameter 710 to aid in capturing all of the light from the proximal fiber (e.g., first section 704), thereby promoting increased attenuation, which may enable transmission of the light from the light along the light fiber 700 with minimal attenuation (e.g., only inherent attenuation due to material properties of the fibers). It will be recognized that a smaller difference in diameter between first diameter 708 and second diameter 710 increases the chance of mismatching the core (e.g., core not aligned), thereby increasing the loss of light transmission.

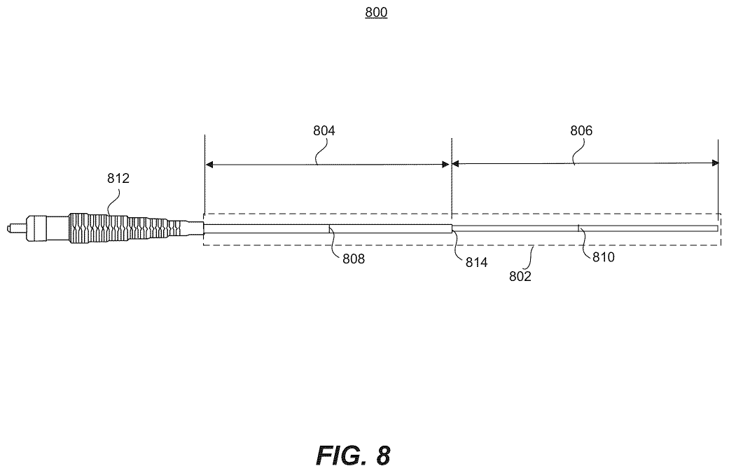

FIG. 8 illustrates an example of a light fiber, consistent with embodiments of the present disclosure. Light fiber 800 may include fiber optic cables 802 having a first section 804, which may be disposed proximal to a light source connector 812, and a second section 806, which may be disposed distal to the light source connector 812. In some examples, the first section 804 may abut the second section 806. The first section 804 may have a first diameter 808, and the second section 806 may have a second diameter 810. In an example, the first diameter 808 may be greater than the second diameter 810. As such, light fiber 800 may have a loss of light transmitted from light source connector 812 to an emission zone disposed distal from the light source. For example, due to the light traveling from a larger first diameter 808 to a smaller second diameter 810, light may be lost at the junction 814 where the first section 804 and the second section 806 contact each other. As a result of the difference in diameters, there may be a loss of light at the junction 814 such that some light may not be transferred from the first section 804 to the second section 806. In some embodiments, the first section 804 may be made of a first material, and the second section may be made of a second material. In some examples, the first material may be different than the second material, such as an example in which the first material in section 804 may be silica, and the second material may be PMMA.

It will be recognized that in comparison to the exemplary configuration of light fiber 800 in FIG. 8, the light fiber 700 in FIG. 7 involves a transfer from light from a section with a smaller first diameter 708 to a larger second diameter 710 such that there may be minimal loss of light at the junction between the first section 704 and the second section 706 (e.g., the light may be captured during transfer), whereas the difference between the larger first diameter 808 and the smaller second diameter 810 may prevent some light from being captured and transferred to the second section 806 (e.g., some light escapes at the junction 814 as light transfers from the first section 804 to the second section 806). It will be appreciated that by changing the difference in size between the first diameter 808 and the second diameter 810, the loss in light at the junction 814 may be adjusted, thereby varying the amount of light output at an emission zone that may be disposed within the second section 806. For example, increasing a difference in the diameters may result in a larger loss of light. Increasing a difference between the diameters may refer to increasing a diameter of a fiber, decreasing the diameter of a fiber, or both increasing a diameter of a fiber and decreasing the diameter of a second fiber. As such, the difference between first diameter 808 and second diameter 810 may be predetermined to modulate a predicted loss of the light output.

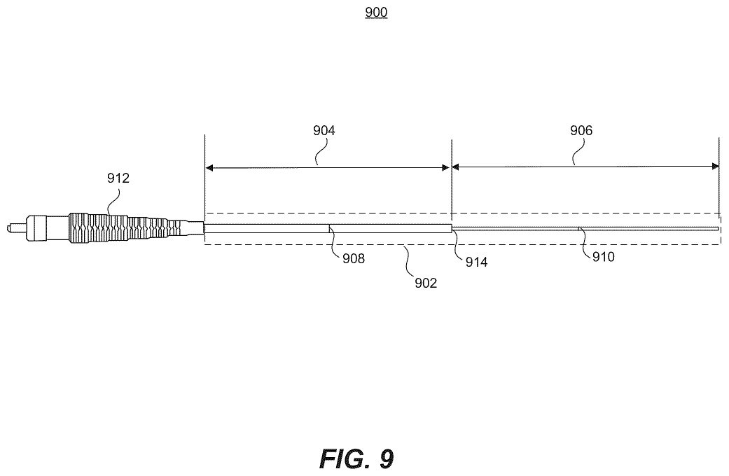

FIG. 9 illustrates a light fiber, consistent with embodiments of the present disclosure. Light fiber 900 may include an optic cable 902 having a first section 904, which may be disposed proximal to a light source connector 912, and a second section 906, which may be disposed distal to the light source connector 912. In some examples, the first section 904 may abut the second section 906. The first section 904 may have a first diameter 908, and the second section 906 may have a second diameter 910. In an example, the first diameter 908 may be greater than the second diameter 910. As such, light fiber 900 may have a loss of light transmitted from light source connector 912 to an emission zone disposed distal from the light source. For example, due to the light traveling from a larger first diameter 908 to a smaller second diameter 910, light may be lost at the junction 914 where the first section 904 and the second section 906 contact each other. In an example, the difference between first diameter 908 and second diameter 910 may be larger than the difference between first diameter 808 and second diameter 810 as referenced in FIG. 8, thereby resulting in light fiber 900 having an increased loss of light output than light fiber 800. As such, predetermining (e.g., before delivery to the target lumen) the difference in the diameters of a light fiber may enable increasing or decreasing a predicted loss of the light output. It will be appreciated that light fiber 900 may include additional sections having various diameters, such as a third section, to further modulate the loss in light output.

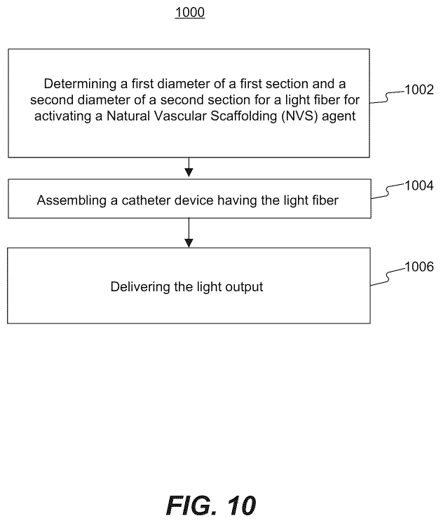

FIG. 10 illustrates an exemplary flow diagram of a method for configuring a light output, consistent with embodiments of the present disclosure. Method 1000 may include a step 1002 of determining a first diameter of a first section and determining a second diameter of a second section for a light fiber, as described herein. For example, Step 1002 may involve calculating the amount of light needed to activate the NVS for a given vessel size. As described herein, the amount of light may be the minimum needed to activate the NVS, accounting for any distance between the light output and the light fiber. Determining the first diameter and the second diameter may involve selecting the diameter(s) to achieve the desired light output (e.g., selecting diameters which may cause a loss in light transmission), as described herein. In some embodiments, determining the first diameter and the second diameter may involve selecting or predetermining a difference between diameters of different sections in order to achieve a predicted or expected light output. For example, multiple light fibers could be configured with varying differences between diameters of different sections, and the expected light output could be measured based on the difference between the diameters for the light fibers, enabling the generation of a formula or an algorithm to predict the expected light output (e.g., loss of light transmission). As described herein, the difference in the diameter may be increased or decreased by increasing and/or decreasing diameters of different sections, such as when the diameter of the second section may be different than the diameter of the first section. In an example, the determination of diameters may refer to selecting diameters for the first section and the second section, including selecting different materials for the first and second sections. Method 1000 may include a step 1004 of assembling or manufacturing a catheter device having a light fiber for activating a natural vascular scaffolding (NVS) agent, the light fiber extending distally from the proximal end to an emission zone, as described herein. Step 1004 may involve assembling the catheter device such that the light fiber includes a first section with a first diameter and a second section with a second diameter, with the difference between the diameters enabling the variation of the light output. Method 1000 may include a step 1006 of delivering (e.g., providing) the light output. In use, step 1006 may involve providing the catheter to a target location and delivering the light output at the emission zone of the light fiber. As such, the light output achieved may depend on the diameters selected in step 1002 (e.g., the diameters may be selected before delivery of the catheter). For example, step 1002 may involve increasing the difference, thereby increasing the predicted loss of light. In another example, step 1002 may involve decreasing the difference, thereby reducing the predicted loss of light.

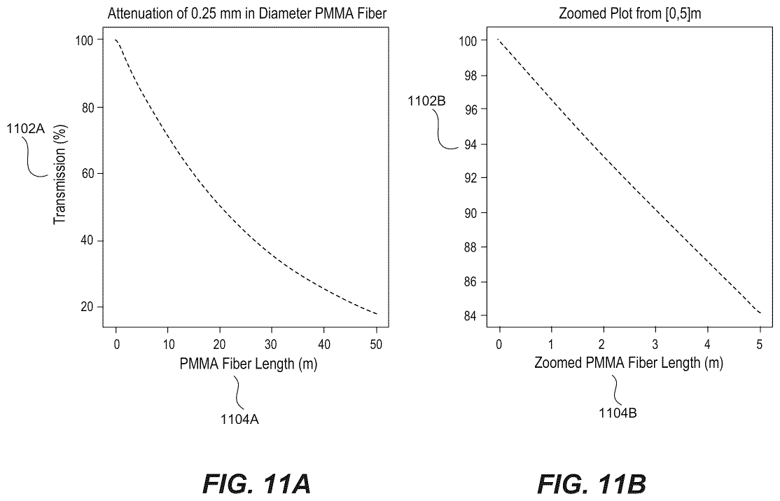

As described herein, attenuation in a sterilized light fiber may depend on length and/or core diameter of the light fiber. In an example, the attenuation of a PMMA fiber at 450 nm may be 0.1 dB for a 500 μm core diameter fiber, and the attenuation may be 0.15 dB for a 250 μm core fiber, holding the wavelength of the light and the length of the fiber constant. The attenuation may increase as the core diameter decreases, and the attenuation may decrease as the diameter increases. Similarly, for a given constant diameter, changing materials from PMMA to silica may decrease the attenuation. Attenuation may be defined as dB=10 log (I₀/I)/x, where T (e.g., transmission)=I₀/I and x may be the position in meters. As such, in an example, the transmission of a 250 μm core PMMA fiber of 0.15 dB at 450 nm wavelength may be 96.6% transmission for a light fiber having a one meter length. Similarly, if an exemplary light fiber was two meters, long the transmission may be 93.3%. As such it will be recognized that, as the length of the fiber increases, there may be more attenuation (e.g., more transmission loss), and therefore less output light available. FIGS. 11A-11B are graphs of exemplary light attenuation in a light fiber, consistent with embodiment of the present disclosure. Referring to FIG. 11A, it will be recognized that the transmission 1102A of the light output decreases as the length 1104A of the light fiber increases, as described herein. FIG. 11B illustrates a zoomed in version of FIG. 11A, indicating that to reduce the transmission 1102B by 10% (100% to 90%), the length 1104B of the exemplary light fiber may need to be increased by three meters. As such, it will be appreciated that the disclosed embodiments may enable a more efficient method of reducing the light output to achieve a desired clinical application.

Sections of light fibers, including first and second sections as described herein, may be disposed coaxial with one another in some examples. For example, the core of the first section may be flush or substantially aligned with the core of a second section such that the sections share a common axis, thereby maintaining transmission of the light. In some examples, the sections may be misaligned, such that the first section may be off-axis with respect to the second section, thereby creating the potential for losses of light transmission. Some disclosed embodiments may involve sections which do not abut. For example, a first section and a second section of a light fiber as described herein may be spaced apart, such as by an air gap, thereby resulting in transmission losses due to the non-continuity between sections of light fiber material. At the gap, light may be lost and/or scattered. It will be recognized that a larger spacing between sections of material (e.g., a larger air gap) may result in larger losses of light transmission. In some examples, a light fiber may have multiple air gaps to further modulate the transmission of light.

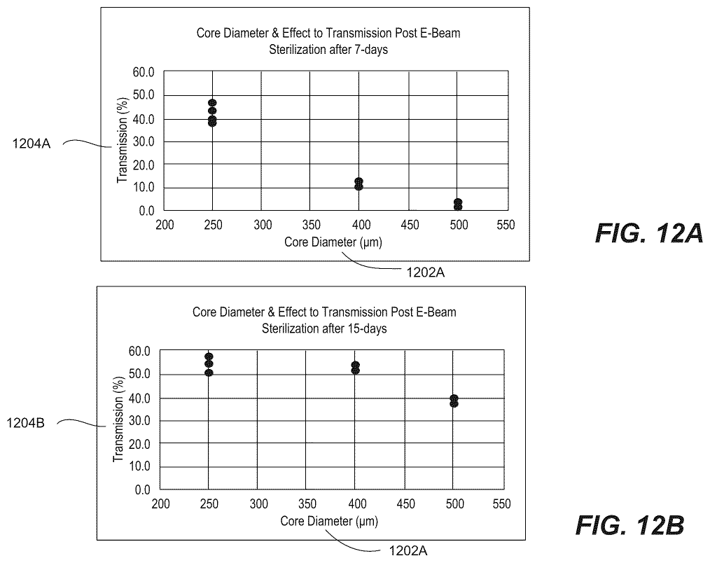

In some embodiments, the diameter of a light fiber may affect the effect of electron beam sterilization on light fiber materials and their performance. For materials configured for electron beam sterilization as described herein, increasing the diameter of the light fiber may result in reducing the effect of the electron beam sterilization. Some disclosed embodiments involve predetermining a diameter of the light fiber to modulate the effect of the electron beam sterilization on the light transmission. For example, the diameter of the light fiber (such as diameter 708 or any diameter described herein) may be determined before the application of electron beam sterilization. FIGS. 12A-12B illustrate graphs of the effect of core diameter for light fibers including PMMA, consistent with embodiments of the present disclosure. Particularly, FIGS. 12A-12B illustrate how the diameter of a fully-PMMA light fiber influences the effect of electron beam sterilization on the transmission seven days after electron beam sterilization (FIG. 12A) and 15 days after electron beam sterilization (FIG. 12B) for a light fiber including PMMA. As illustrated, the smaller core diameters represented on axis 1202A, 1202B have a decreased effect of the electron beam sterilization, demonstrated by a smaller difference between transmission 1204A at seven days and transmission 1204B at 15 days. In some embodiments, the difference between the transmissions 1204A at seven days and 1204B at 15 days for a 250 micrometer fiber may be smaller than the difference between the transmissions 1204A at seven days and 1204B at 15 days for a 600 micrometer fiber, as illustrated in FIGS. 12A-12B. Thus, for light fibers configured for electron beam sterilization (e.g., including materials such as PMMA), decreasing the diameter may lessen the effect of the electron beam sterilization. Thereby, decreasing the diameter may reduce the reduction in transmission of the light output (e.g., the effect of the electron beam sterilization), resulting in an increase in the transmission (e.g., an increase in transmissibility over time or after a certain time period). Similarly, increasing the diameter may increase the effect of the electron beam sterilization for some materials (e.g., PMMA), thereby increasing the reduction of transmission. In some examples, the reduction in transmission may occur after a certain time period, such as a few days or a few weeks. Thus, increasing the core diameter may result in a desired reduction of light output in examples in which the light fiber has been electron beam sterilized. The diameter may be predetermined to obtain the desired modulation of the effect of the electron beam (and therefore the change in the transmission of the light). For example, to obtain a reduction of 10% in transmission after a time period of 15 days (e.g., 50% to 40%), the light fiber diameter may be selected such that the diameter increases by 250 micrometers, as referenced in FIGS. 12A-12B.

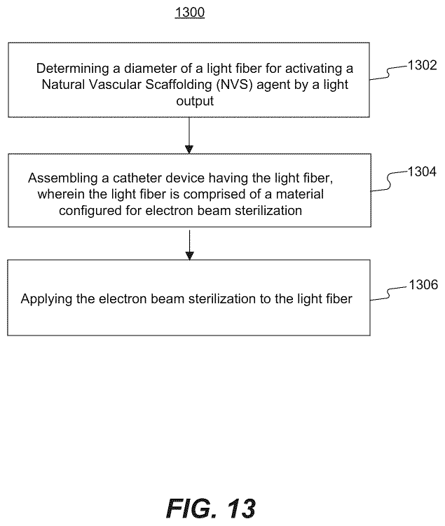

FIG. 13 illustrates an exemplary flow diagram of a method for configuring a light output, consistent with embodiments of the present disclosure. Method 1300 may involve a step 1302 of determining a diameter of a light fiber for activating a NVS agent by a light output. Step 1302 may involve determining the diameter, as described herein, such that the light fiber delivers an expected or predicted light output (e.g., at the emission zone). For example, the diameter of the light fiber may be selected to achieve a desired light output in use as described herein, including through experimental methods, formulas, or algorithms. As described herein, the diameter of the light fiber may be determined to modulate a predicted reduction in transmission of the light output due to the electron beam sterilization. Method 1300 may involve a step 1304 of assembling or manufacturing a catheter device having the light fiber. The light fiber may include a material configured for electron beam sterilization, such as PMMA, and the light fiber may be assembled to include the determined diameter such that the desired light output may be achieved after electron beam sterilization. Method 1300 may also involve one or more sections each having a different diameter or being comprised of different materials Method 1300 may include as step 1306 of applying the electron beam sterilization. As such, method 1300 may involve an increase or a decrease in the reduction of the light transmission depending on the predetermined diameter. In some examples, the effect of the electron beam sterilization may occur after a time period in step 1306. For example, the light fiber may be used to apply NVS therapy a week, two weeks, or a longer time period after the electron beam sterilization has been applied. Method 1300 may also involve providing, in use, the catheter to the target location for delivery of the light output. As such, in use, the light output may activate the NVS agent.

Accordingly, the apparatus and methods described herein provide the delivery of NVS to a treatment area (e.g. a vessel) and provide restoration to that treatment area using the apparatus or according to the methods described above. Disclosed embodiments of varying the light output through modifications to the light fiber may prevent errors associated with modifying the power of the light source itself, as well as enable cost savings compared to modifying the light source itself. The apparatus and method described above provide concurrently scoring the vessel, treating the vessel with one or more drugs (e.g. with Paclitaxel and/or NVS) with minimal loss to other vessels, scaffolding and casting the vessel, and light activation of the one or more drugs delivered to the treatment area. Further, it will be appreciated that disclosed embodiments include providing Natural Vascular Scaffolding (NVS), which may be advantageous over other systems and methods as NVS may not require metal stents which are left behind in the vessel after the procedure. One advantage may include the reduction of restenosis. These advantages can be accomplished utilizing the apparatus and methods described herein.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A catheter device comprising:
a catheter body having a proximal and a distal end, the catheter body having a light fiber for activating a natural vascular scaffolding (NVS) agent, the one or more light fibers extending distally from the proximal end to an emission zone,
a first section of the light fiber comprised of a first material extending a first length;
a second section of the light fiber comprised of a second material extending a second length, wherein the second material is different than the first material; and
wherein the emission zone is disposed proximal to the distal end.

2. The device of claim 1, wherein the first material comprises silica.

3. The device of claim 1, wherein the second material comprises Polymethyl Methacrylate (PMMA).

4. The device of claim 1, wherein the emission zone is disposed within the second section.

5. The device of claim 1, wherein the device is configured for electron beam sterilization.

6. The device of claim 5, wherein a change of the first length or the second length modulates a light output.

7. The device of claim 6, wherein the emission zone is configured so that, in use, light output activates the natural vascular scaffolding agent.

8. The device of claim 1, wherein the first section and the second section abut each other.

9. The device of claim 1, further comprising a third section extending a third length, wherein the third section is comprised of a third material, wherein the third material is different than the second material.

10. The device of claim 1, further comprising a light source for the light output, wherein the light source is coupled to the proximal end.

11. A method for configuring a light output of a light fiber device, the method comprising:
determining, for a light fiber for activating a natural vascular scaffolding (NVS) agent, a first length of a first section comprised of a first material and a second length of a second section comprised of a second material;
assembling a catheter device comprising:
a body having:
a proximal and a distal end; and
the light fiber, the light fiber extending distally from the proximal end to an emission zone,
wherein the second material is different than the first material; and
wherein the emission zone is disposed proximal to the distal end; and
sterilizing the catheter device with electron beam sterilization, wherein the determined first length or the determined second length varies a light output of the light fiber.

12. The method of claim 11, wherein the first material comprises a material not affected by electron beam sterilization.

13. The method of claim 11, wherein the first material comprises silica.

14. The method of claim 13, further comprising increasing the first length, wherein increasing the first length reduces a loss of light output after electron beam sterilization.

15. The method of claim 11, wherein the second material comprises Polymethyl Methacrylate (PMMA).

16. The method of claim 15, further comprising increasing the second length, wherein increasing the second length reduces the light output.

17. The method of claim 16, wherein reducing the light output modulates the activation of the natural vascular scaffolding.

18. The method of claim 11, wherein the emission zone is disposed within the second section.

19. The method of claim 11, wherein the first section and the second section abut each other.

20. The method of claim 11, further comprising a third section extending a third length, wherein the third section is comprised of a third material, wherein the third material is different than the second material.
